(19) United States
(12) Patent Application Publication
(10) Pub. No.: US 2023/0357848 A1
(43) Pub. Date: Nov. 9, 2023

(54) PRIMER PAIR FOR IDENTIFYING GENE SEGMENT OF MALE-SPECIFIC MOLECULAR MARKER OF AMERICAN SHAD AND USES THEREOF

(71) Applicant: Suzhou Fishseeds Bio-Technology Co., Ltd., Suzhou (CN)

(72) Inventors: Jia DU, Suzhou (CN); Qinghua LIU, Suzhou (CN)

(21) Appl. No.: 18/149,681

(22) Filed: Jan. 4, 2023

(30) Foreign Application Priority Data
May 5, 2022 (CN) 202210478184.7

(51) Int. Cl. C12Q 1/6879

(52) U.S. Cl. CPC C12Q 1/6879; C12Q 2600/158

(57) ABSTRACT

The disclosure provides a primer pair for identifying a gene segment of a male-specific molecular marker of American shad (Alosa sapidissima). The gene segment has a nucleic acid sequence represented by SEQ ID NO: 2, and the primer pair has nucleic acid sequences represented by SEQ ID NO: 3 and SEQ ID NO: 4.

### CROSS-REFERENCE TO RELATED APPLICATIONS

Pursuant to 35 U.S.C.§ 119 and the Paris Convention Treaty, this application claims foreign priority to Chinese Patent Application No. 202210478184.7 filed May 5, 2022, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### INCORPORATION-BY-REFERENCE OF SEQUENCE LISTING

[0001.1] This application contains a sequence listing, which has been submitted electronically in XML file and is incorporated herein by reference in its entirety. The XML file, created on Dec. 14, 2022, is named SZFS-00401-UUS.xml, and is 29,880 bytes in size.

### BACKGROUND

The disclosure related to the field of fisheries genetics, and more particularly, to a specific primer pair for identifying a gene segment of male-specific molecular marker of American shad and uses thereof.

The growth speed of males and females of American shad (Alosa sapidissima) differs from each other greatly, with males growing 32.4% slower than females (see FIG. 1) and being identified as troublemakers during shad farming. In spring, 1 to 2-year-old male shad will harass females by chasing behaviors that causes mild injuries to the females, including physical damage, scale loss, and even death. The harassment consumes much energy of the female shad, leading to weight loss and high mortality rate, thereby reducing the production of shad and restricting the growth of the fishing industry. All-female population may grow fast in the absence of the male shad, which reduces costs caused by the harassment, thereby increasing the survival rate and growth of shad and improving profitably.

The all-female population can be developed using sex-reversed male (XX neomales). The XX neomales are phenotypically male but genotypically female; specifically, sex hormone is used and causes sex reversal from female to male phenotype having mature testes; the XX neomales are crossed with normal females to produce XX females. Sex reversal is performed by feeding the juvenile with androgen to produce the XX neomales and XY females, which have the same physical appearance, and thus a male-specific DNA molecular marker is necessary for distinguishing the XX neomales from the XY females. The XX neomales are crossed with the normal females (XX) to produce the all-female populations. Therefore, screening for and applying male-specific DNA markers is critical to the production of all-female shad.

Currently, sex-specific genetic markers are concentrated primarily in salmon, tilapia, and a few carps. There have been no reports of shad and similar herrings having sex-specific markers.

### SUMMARY

The first objective of the disclosure is to provide a male-specific molecular marker of American shad (Alosa sapidissima), the male-specific molecular marker comprising a first nucleic acid sequence of SEQ ID NO: 1.

The second objective of the disclosure is to provide a gene segment represented by a second nucleic acid sequence of SEQ ID NO: 2 comprising the first nucleic acid sequence.

The third objective of the disclosure is to provide a primer pair represented by SEQ ID NO: 3 and SEQ ID NO: 4 for identifying the male-specific molecular marker or the gene segment.

The fourth objective of the disclosure is to provide a method for preparing the male-specific molecular marker, and the method comprises:
1) extracting total DNA from caudal fins of male and female American shad;
2) preparing a gene/DNA library of American shad using 2b-RAD method; checking the quality of the library; and sequencing the qualified library; and
3) filtering-out unsuitable sequencing data; and searching the first nucleic acid sequence of SEQ ID NO: 1, i.e., the male-specific molecular marker.

The fifth objective of the disclosure is to provide a method for preparing the second nucleic acid sequence; the method comprises above steps 1) - 3) and further comprises 4): aligning the first nucleic acid sequence to a genome sequence (accession number JAHTKL010000000) of American shad by Basic Local Alignment Search Tool (BLAST) in the NCBI sequencing database to obtain the second nucleic acid sequence of SEQ ID NO: 2.

The sixth objective of the disclosure is to provide a method for preparing a kit for determining a genetic sex of American shad comprising applying the primer pair.

The seventh objective of the disclosure is to provide a kit for determining the genetic sex of American shad; and the kit comprises the primer pair.

The kit further comprises a DNA extraction reagent, a PCR amplification reagent, a molecular marker (i.e., a DNA marker), a nucleic acid stain, or a combination thereof; the nucleic acid stain includes, but is not limited to DNA stain (SYBR Safe).

The eighth objective of the disclosure is to provide a method for determining the genetic sex of American shad by use of the DNA marker; and the method comprises:
1) extracting total DNA of American shad of unknown sex;
2) amplifying a target fragment of the total DNA through a PCR reaction with the primer pair or the kit; and
3) determining, when an amplified fragment is 470 bp in length, the American shad is male; otherwise, the American shad is female.

A PCR system for the PCR reaction comprises a 2×Taq reaction mixture II (2×Taq Plus Master Mix II (Dye Plus)), the primer pair, a template DNA and H₂O; PCR cycling and reaction parameters are detailed as follows: initial denaturation at 95° C. for 3 min, denaturation at 95° C. for 15 s, annealing at 60° C. for 30 s, elongation at 72° C. for 1 min, and final extension at 72° C. for 5 min.

In a class of this embodiment, the total DNA is extracted from caudal fins of five female American shad and five male American shad; the library is prepared using the 2b-RAD method; the quality of the library is checked; the qualified library is sequenced; the resulting raw data is presented in fastq format, cleaned and clustered to build a reference sequence using Ustacks software (version 1.34) in Stacks; the sequencing data is aligned to the reference sequence using SOAP software (version 2.2), and single nucleotide polymorphism (SNP) calling is performed using the maximum likelihood (ML); SNP markers are identified for sex identification; a short sequence including the SNP markers is found in the five male American shad but not in the five female American shad; and the short sequence is the male-specific molecular marker comprising the first nucleic acid sequence of SEQ ID NO: 1.

In a class of this embodiment, the first nucleic acid sequence is 27 bp in length and not long enough for use in primer design; the first nucleic acid sequence is aligned to the genome sequence (accession number JAHTKL010000000) of American shad by BLAST in the NCBI sequencing database to obtain the second nucleic acid sequence of SEQ ID NO: 2; the second nucleic acid sequence is 470 bp in length.

In a class of this embodiment, the second nucleic acid sequence is employed as a template sequence to design a primer pair Tag-5 comprising two short nucleic acid sequence of SEQ ID NO: 3 and SEQ ID NO: 4.

The disclosure uses the RAD sequencing to provide the male-specific molecular marker and the primer pair thereof for sex determination. Identification of XX neomales is critical to the propagation of an all-female strain of American shad, thereby increasing the profitably.

The following advantages are associated with the disclosure: the disclosure provides a male-specific molecular marker, a primer pair and a kit thereof to distinguish the genotypically female (XX neomales) from the phenotypically male; the disclosure further provides a method for determining the genetic sex of American shad by use of the male-specific molecular marker. Total DNA is extracted from caudal fins of American shad at the fry or juvenile stage to determine sex without causing injury. The primer pair achieved a 100% accuracy rate in determining the sex of a large number of American shad using high-throughput DNA sequencing.

### BRIEF DESCRIPTION OF THE DRAWINGS

FIG. 1 is a graph showing average growth rate of male and female American fish;

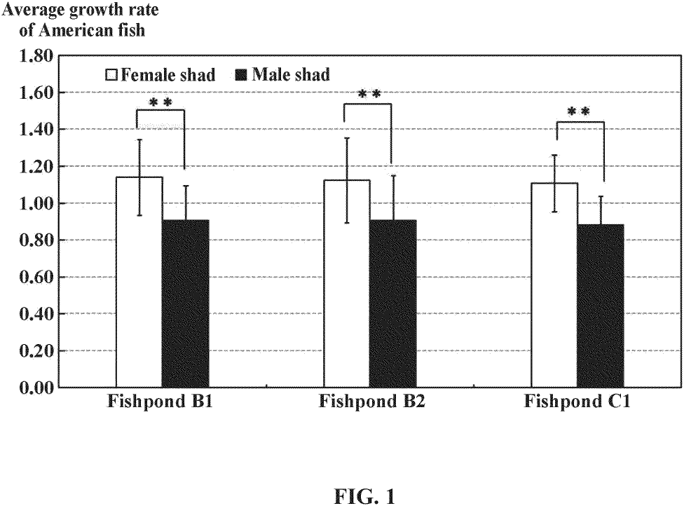

FIG. 2 shows the results of genetic sex determination in American shad using a primer pair Tag-5; and

FIG. 3 shows the results of genetic sex determination and validation of a male-specific molecular marker in the population of American shad.

### DETAILED DESCRIPTION OF THE EMBODIMENTS

The disclosure uses Restriction-site associated DNA (RAD) sequencing to provide a male-specific molecular marker for sex determination in American shad; the male-specific molecular marker is elongated within a reference genome of American shad to form a second nucleic acid sequence for primer design.

The RAD sequencing is a technique that uses restriction enzymes to isolate a RAD Tag that is employed as a template and analyzed through use of high-throughput DNA sequencing to generate a large volume of raw sequence reads for preparation of a library. The RAD technique is a simple, stable and efficient genotyping method that reduces the complexity of a genome.

### Example 1 Identification of a Male-Specific Molecular Marker of American Shad

The method involves DNA extraction, restriction enzyme digestion, ligation, enrichment, purification, introduction of an index sequence by PCR, pooling and sequencing.

Total DNA was extracted from caudal fins of five female and five male American shad of known sex using a Marine Animals DNA Kit (Catalog number: DP324-03, TIANGEN);

absolute ethanol was added to a buffer GD and a rinse solution PW (see the bottle label for volume); specifically, the DNA extraction method comprises the following steps:
(1) no more than 30 mg of tissue samples was taken, frozen in liquid nitrogen, ground into a fine powder, and transferred to a centrifuge tube containing 200 µL of a GA buffer; the mixture was vortexed for 15 s; to remove RNA from DNA extraction, 4 µL of 100 mg/mL ribonuclease (RNase) A solution was added, shaken for 15 s, and allowed to stand at room temperature for 5 min;
(2) 20 µL of 20 mg/mL proteinase K solution was added and vortexed; the centrifuge tube was centrifuged to remove drops from the inside of the cover; the mixture was allowed to stand at 56° C. until the tissue is completely dissolved; the mixture was centrifuged to remove water droplets on the inner wall of the centrifuge tube cover;
(3) 200 µL of a buffer GB was added, mixed thoroughly by inversion, allowed to stand at 70° C. for 10 min; when the mixture turned clear, the centrifuge tube is centrifuged to remove drops from the inside of the cover;
(4) 200 µL of absolute ethanol was added, mixed thoroughly by inversion, and a flocculent precipitate may form; the centrifuge tube was centrifuged to remove drops from the inside of the cover;
(5) the resulting mixture containing the flocculent precipitate in 4) were added into an adsorption column CB3 (placed in a collection tube), centrifuged at 12,000 rpm (-13,400×g) for 30 s; the waste liquid was discarded; and the adsorption column CB3 is placed back into the collection tube;
(6) 500 µL of the buffer GD was added to the adsorption column CB3 (make sure the absolute ethanol has been added before use), centrifuged at 12,000 rpm (-13,400×g) for 30 s; the waste liquid was discarded, and the adsorption column CB3 placed back into the collection tube;
(7) 600 µL of the rinse solution PW was added to the adsorption column CB3 (make sure the absolute ethanol has been added before use), centrifuged at 12,000 rpm (-13,400×g) for 30 s; the waste liquid was discarded, and the adsorption column CB3 placed back into the collection tube;
(8) the step 7) was repeated;
(9) the adsorption column CB3 was placed back into the collection tube, centrifuged at 12,000 rpm (-13,400xg) for 2 min; and the waste liquid was discarded; the adsorption column CB3 was left at room temperature for several minutes, allowing the residual rinse solution PW to evaporate from the adsorption membrane of the adsorption column CB3, with no effect on the subsequent enzyme reaction experiments (such as enzyme digestion and PCR reaction); and
(10) the adsorption column CB3 was placed into a new centrifuge tube; 50-200 µL of an elution buffer TE was suspended in the middle of the adsorption membrane, allowed to stand at room temperature for 2-5 min, and centrifuged at 12,000 rpm (-13,400xg) for 2 min; and the solution was collected in the new centrifuge tube.

Note: less than 50 µL of the elution buffer TE added to the adsorption membrane results in low DNA recovered yields. For larger DNA yields, after centrifugation, the solution was added back into the adsorption column CB3, allowed to stand at room temperature for 2 min, and centrifuged at 12,000 rpm (-13,400xg) for 2 min; the pH value of the elution buffer TE was an important influence on the elution efficiency; for example, the elution efficiency is reduced when the DNA product was eluted with ddH₂O with a pH value of 7.0-8.5; and the DNA product was stored at -20° C. to prevent DNA degradation.

### 2. Restriction Enzyme Digestion

In the laboratory, five female and five male shad of known sex were used to prepare a library using the 2b-RAD method. Specifically, the restriction enzyme digestion reaction contained the components listed in Table 1 and took place at 37° C. for 45 min.

TABLE 1

| Component | Volume (µL/sample) |
|---|---|
| DNA (200 ng/µL) | 1 |
| 10×CutSmart buffer | 1.5 |
| BsaXI restriction endonuclease (2U/µL) | 0.5 |
| Pure water | 12 |
| Total volume | 15 |

4 µL of the digested product was loaded on 1% (wt/vol) agarose gel and electrophoresed at 100 V for 10-15 min; and the bands were visualized under ultraviolet light.

### 3 Ligation

The components for ligation reaction was listed in Table 2; five samples were labeled as 1-5, respectively. According to Table 3, two adaptors (A and B) were added to a corresponding sample (Ten adaptors (Ada 1-10) were prepared by annealing two DNA strands, for example, Ada 1 is prepared from Ada1a and Ada1b, Ada 2 is prepared from Ada2a and Ada2b; the specific sequences of adapters were shown in Table 4); 0.8 µL of each adaptor was added and ligated to the corresponding end of the digested product at 16° C. for 1 h; and the five ligated products were labeled as 1-5, respectively.

TABLE 2

| Component | Volume (µL/ sample) |
|---|---|
| Digested product | 10 |
| 10× T4 ligase buffer | 1 |
| 10 mM adenosine triphosphate (ATP) | 1 |
| Adaptor A (5 µM) | 0.8 |
| Adaptor B (5 µM) | 0.8 |
| T4 DNA ligase (400 U/µL) | 0.5 |
| Pure water | 5.9 |
| Total volume | 20 |

TABLE 3

Five groups of adaptors

| Label | Adaptor A (5 µM) | Adaptor B (5 µM) |
|---|---|---|
| 1 | Ada 1 | Ada 2 |
| 2 | Ada 3 | Ada 4 |
| 3 | Ada 5 | Ada 6 |
| 4 | Ada 7 | Ada 8 |
| 5 | Ada 9 | Ada 10 |

TABLE 4

Oligonucleotide sequences of adapters and primers
Sequence (5′ - 3′)
Adaptor

| | | |
|---|---|---|
| SEQ ID NO: 5 | Ada1a | ACACTCTTTCCCTACACGACGCTGTTCCGATCTN |
| SEQ ID NO: 6 | Ada1b | AGATCGGAACAGC |
| SEQ ID NO: 7 | Ada2a | GTGACTGGAGTTCAGACGTGTGCTCTTCACGAN |
| SEQ ID NO: 8 | Ada2b | TCGTGAAGAGCAC |
| SEQ ID NO: 9 | Ada3a | ACACTCTTTCCCTACACGACGCTCTTCATCGNNN |
| SEQ ID NO: 10 | Ada3b | CGATGAAGAGCGT |
| SEQ ID NO: 11 | Ada4a | GTGACTGGAGTTCAGACGTGTGCTCTTCAGCAN |
| SEQ ID NO: 12 | Ada4b | TGCTGAAGAGCAC |
| SEQ ID NO: 13 | Ada5a | ACACTCTTTCCCTACACGACGCTCTTCATGCNNN |
| SEQ ID NO: 14 | Ada5b | GCATGAAGAGCGT |
| SEQ ID NO: 15 | Ada6a | GTGACTGGAGTTCAGACGTGTGCTCTTCAGACN |
| SEQ ID NO: 16 | Ada6b | GTCTGAAGAGCAC |
| SEQ ID NO: 17 | Ada7a | ACACTCTTTCCCTACACGACGCTCTTCAGTCNNN |
| SEQ ID NO: 18 | Ada7b | GACTGAAGAGCGT |
| SEQ ID NO: 19 | Ada8a | GTGACTGGAGTTCAGACGTGTGCTCTTCACAGN |
| SEQ ID NO: 20 | Ada8b | CTGTGAAGAGCAC |
| SEQ ID NO: 21 | Ada9a | ACACTCTTTCCCTACACGACGCTCTTCACTGNNN |
| SEQ ID NO: 22 | Ada9b | CAGTGAAGAGCGT |
| SEQ ID NO: 23 | Ada10a | GTGACTGGAGTTCAGACGTGTGCTGTTCCGATC |
| SEQ ID NO: 24 | Ada10b | AGATCGGAACAGC |
| SEQ ID NO: 25 | Prim1 | ACACTCTTTCCCTACACGACGCT |
| SEQ ID NO: 26 | Prim2 | GTGACTGGAGTTCAGACGTGTGCT |
| SEQ ID NO: 27 | BioPrim1 | (biotin)-ACACTCTTTCCCTACACGACGCT |
| SEQ ID NO: 28 | BioPrim2 | (biotin)-GTGACTGGAGTTCAGACGTGTGCT |
| SEQ ID NO: 29 | I5 index primer | AATGATACGGCGACCACCGAGATCTACACNNN NNNACACTCTTTCCCTACACGACGCTCTTCCGAT |
| SEQ ID NO: 30 | I7 index primer | CAAGCAGAAGACGGCATACGAGATNNNNNNGT GACTGGAGTTCAGACGTGTGCTCTTCCGATCT |

### 4. Enrichment and Purification

(1) The components for PCR reaction were listed in Table 5; referring to Table 6, the ligated products were amplified using a corresponding primer pair labeled with the same number (the specific sequences of primers were shown in Table 4);

TABLE 5

| Component | Volume (µL/ sample) |
|---|---|
| Ligated product | 18 |
| 5× Phusion HF buffer | 10 |
| 10 Mm deoxyribonucleoside triphosphate (dNTP) | 1.2 |
| Primer A (10 µM ) | 0.8 |
| Primer B (10 µM ) | 0.8 |
| Phusion high-fidelity DNA polymerase (2 U/µ adaptor) | 0.4 |
| Pure water | 18.8 |
| Total volume | 50 |

TABLE 6

Primer pairs for five indexes

| Index position | Primer A (10 µM ) | Primer B (10 µM ) |
|---|---|---|
| 1 | Prim 1 | BioPrim2 |
| 2 | BioPrim1 | BioPrim2 |
| 3 | BioPrim1 | BioPrim2 |
| 4 | BioPrim1 | BioPrim2 |
| 5 | BioPrim1 | Prim 2 |

(2) PCR cycling and reaction parameters:

TABLE 7

| Cycles | Denaturation | Annealing | Elongation |
|---|---|---|---|
| 1-16 | 98°C, 5 s | 60° C., 20 s | 72° C., 10 s |

(3) 50 µL of PCR product and 1 µL of 100-bp DNA ladder (Takara, cat. no. 3427) were loaded on 8% polyacrylamide gel and electrophoresed at 400V for 35 min;
(4) the 8% polyacrylamide gel was then stained with a nucleic acid stain (SYBR Safe) for 3 min; and the brightness of the target band (with a size of 100 bp) was observed;
(5) an agarose slice with the band of interest was cut, placed into a 1.5 mL centrifuge tube, ground with a grinding rod; 30-40 µL of pure water was added and allowed to stand at 37° C. for 30 min;
(6) the mixture was centrifuged at 14000 g at room temperature for 2 min, and a PCR product was recovered; to increase the PCR product yield, 12 µL of each recovered product was used as a template for use in PCR amplification; the PCR procedure was the same as in 2), and the number of cycles was reduced to 4-6;
(7) the enriched products obtained in 6) was mixed and then purified with MinElute PCR purification kit; 15 µL of pure water was added to elute the enriched products; the purified product was quantified with a NanoVue spectrophotometer; preferably, the concentration of the purified product was 10-30 ng/µL;

(8) the enzyme digestion reaction was prepared according to Table 8 and took place at 37° C. for 30 min;

TABLE 8

| Component | Volume (µL) |
|---|---|
| Purified product | 10 |
| 10×CutSmart buffer | 3 |
| 10 Mm adenosine triphosphate (ATP) | 3 |
| SapI restriction endonuclease (10 U/µL) | 0.2 |
| Pure water | 13.8 |
| Total volume | 30 |

(9) 30 µL of the digested product obtained in 8) was added to 10 µL of magnetic beads prepared in advance, and pipetted up and down at room temperature for 5 min;
(10) the centrifuge tube was placed on the magnetic stand; when the digested product was clarified, the supernatant was transferred to a new centrifuge tube; 0.5µL of 400 U/µL T4 DNA ligase was added and allowed to react at 16° C. for 45 min;
(11) the ligated product obtained in 10) was electrophoresed; an agarose slice with the band of interest (with a size of 244 bp) was cut and centrifuged at 14000 g at room temperature for 2 min, so that a pure ligated product was recovered.

### 5. Introduction of an Index Sequence by PCR and Pooling

According to Table 9, the index sequence was introduced by PCR and the specific primer sequences were shown in Table 4.

(1) Components of PCR reaction mixture

TABLE 9

| Components | Volume (µL) |
|---|---|
| Ligated product | 15 |
| 5× Phusion HF buffer | 20 |
| 10 Mm deoxyribonucleoside triphosphate (dNTP) | 2.4 |
| 5 µM I5 index primer | 2 |
| 5 µM I7 index primer | 2 |
| Phusion high-fidelity DNA polymerase (2 U/µL) | 0.8 |
| Pure water | 59.8 |
| Total volume | 100 |

(2) PCR cycling and reaction parameters:

TABLE 10

| Cycles | Denaturation | Annealing | Elongation |
|---|---|---|---|
| 1-16 | 98°C, 5 s | 60° C., 20 s | 72° C., 10 s |

(3) 4µL of PCR products and 1 µL of 100-bp DNA ladder were separately loaded on 1% agarose gel and electrophoresed at 135 V for 10-15 min; and the brightness of the target band (with a size of 299 bp) was observed under ultraviolet light;
(4) the PCR product was purified with MinElute PCR purification kit, eluted with 15 µL pure water and quantified with Qubit; preferably, the concentration of the purified product was 10-30 ng/uL;
(5) if multiple libraries have been built, libraries with different Index numbers were mixed according to the amount of data for sequencing; preferably, the final concentration of the mixed library was 5-10 ng/µL.
(6) the mixed library was sequenced by Illumina PE platform for 100-150 bp paired-end sequencing;
(7) the resulting raw data was presented in fastq format, cleaned and clustered to build a reference sequence using Ustacks software (version 1.34) in Stacks; the sequencing data was aligned to the reference sequence using SOAP software (version 2.2), and SNP calling was performed using the maximum likelihood (ML); SNP markers were identified for sex identification; a short sequence was detected in the five male American shad but not in the five female Ametaican shad; and the short sequence was the male-specific molecular marker comprising a first nucleic acid sequence of SEQ ID NO: 1.

### Example 2 Elongation of a Male-Specific Molecular Marker of American Male Shad

1. The first nucleic acid sequence obtained in Example 1 was aligned to a genome sequence (JAHTKL010000000 in NCBI) of American shad using Blast to obtain a second nucleic acid sequence of SEQ ID NO: 2.

### Example 3 Primer Design and Verification of a Male-Specific Molecular Marker

The second nucleic acid sequence was used as a template sequence to design a primer pair Tag-5 comprising two short nucleic acid sequences of SEQ ID NO: 3 and SEQ ID NO: 4.

Primer sequences for the male-specific molecular marker were listed in Table 11

TABLE 11

Primer sequences for the male-specific molecular marker

| Primer name | Primer sequence (5′-3′) |
|---|---|
| Tag-5 | F: GTTCATTAGTTCCCCTGTGCTGAC |
| | (SEQ ID NO: 31) |
| | R: TCATTATTGGGTTGATAGCAGGCT (SEQ ID NO: 32) |

In the laboratory, total DNA was extracted from 20 American shads (including 10 males and 10 females) of known sex; the DNA extraction method was the same as in Example 1, followed by PCR verification; and the PCR amplification was performed under the conditions:
a 20 µL PCR reaction contained 10 µL of 2 × Taq Plus Master Mix II (Dye Plus), 1 µL of Tag-5-F, 1 µL of Tag-5-R, 1 µL of template DNA, and 7 µL of H₂O;
PCR cycling and reaction parameters were detailed as follows: initial denaturation at 95° C. for 3 min, denaturation at 95° C. for 15 s, annealing at 60° C. for 30 s, elongation at 72° C. for 1 min, and final extension at 72° C. for 5 min.

Preferably, the cycle number was set to 35 cycles.

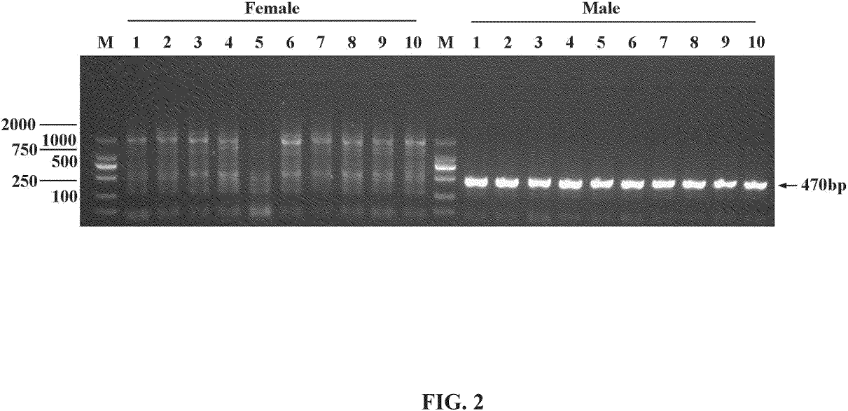

As shown in FIG. 2, the genetic sex of the 20 American shads (comprising 10 males and 10 females) was determined by using the primer pair Tag-5.

### Example 4 Genetic Sex Determination in American Shad by Using the Primer Pair and Verification Thereof

20 female shad and 20 male shad were randomly collected from four shad culture ponds, and subjected to sex verification by using the primer pair for the male-specific molecular marker; the DNA extraction method was the same as in Example 1, followed by PCR verification; the primer sequences were shown in Table 10; and the PCR amplification was performed under the conditions:
a 20 µL PCR reaction contained 10 µL of 2 × Taq Plus Master Mix II (Dye Plus), 1 µL of Tag-5-F, 1 µL of Tag-5-R, 1 µL of template DNA, and 7 µL of H₂O;
PCR cycling and reaction parameters were detailed as follows: initial denaturation at 95° C. for 3 min, denaturation at 95° C. for 15 s, annealing at 60° C. for 30 s, elongation at 72° C. for 1 min, and final extension at 72° C. for 5 min.

Preferably, the cycle number was set to 35 cycles.

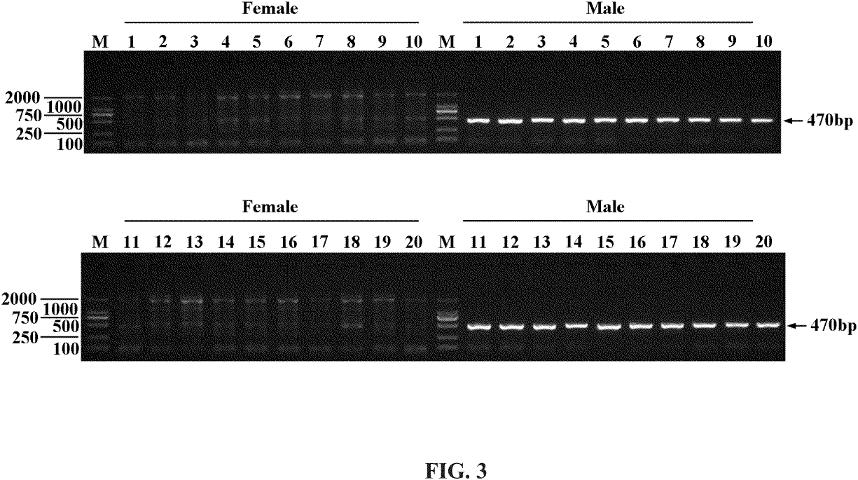

As shown in FIG. 3, the amplified products were electrophoresed on agarose gel; the results showed that the male-specific molecular marker of 470 bp was amplified from the male American shad but not from the female American shad; and the genetic sex of the American shad was verified using the primer pair for the male-specific molecular marker, which was consistent with the results by anatomical gonads, indicating that the disclosed method achieved an accuracy rate of 100%.

It will be obvious to those skilled in the art that changes and modifications may be made, and therefore, the aim in the appended claims is to cover all such changes and modifications.

What is claimed is:

1. A primer pair for identifying a gene segment of a male-specific molecular marker of American shad (Alosa sapidissima), the gene segment having a nucleic acid sequence represented by SEQ ID NO: 2, and the primer pair having nucleic acid sequences represented by SEQ ID NO: 3 and SEQ ID NO: 4.

2. A method for preparing a kit for determining a genetic sex of American shad, the method comprising applying the primer pair of claim 1.

3. A kit for determining a genetic sex of American shad, and the kit comprising the primer pair of claim 1.

4. The kit of claim 3, further comprising a DNA extraction reagent, a PCR amplification reagent, a DNA molecular marker, a nucleic acid stain, or a combination thereof.

5. A method for determining a genetic sex of American shad by use of a DNA marker, the method comprising:
1) extracting total DNA of American shad of unknown sex;
2) amplifying a target fragment of the total DNA through a PCR reaction with the primer pair of claim 1 or the kit of claim 3; and
3) determining, when an amplified fragment is 470 bp in length, the American shad is male; otherwise, the American shad is female.

6. The method of claim 5, wherein a PCR system for the PCR reaction comprises a 2×Taq reaction mixture II (2×Taq Plus Master Mix II (Dye Plus)), the primer pair, a template DNA and H₂O; PCR cycling and reaction parameters are as follows: initial denaturation at 95° C. for 3 min, denaturation at 95° C. for 15 s, annealing at 60° C. for 30 s, elongation at 72° C. for 1 min, and final extension at 72° C. for 5 min.


SEQUENCE LISTING (32 sequences)

SEQ ID NO: 1 - DNA, 27 bp, synthetic construct
  source 1..27: mol_type = other DNA
taaagtgggaggggctgtaatgtacaa

SEQ ID NO: 2 - DNA, 470 bp, synthetic construct
  source 1..470: mol_type = other DNA
gttcattagttcccctgtgctgacctcggccttgtaaaggttatgagctggagatttttttttttttatatgttacactacaaaataaagcttgtcctatgctctttccaataattccagccaaaacttactcatttgagaatttaagcacagagatacatacatatttacagacagggacagacctatttaaatatgacttacataggtctgggacagacctatttacatactgagagaggtgataatggactgatgaactgagtttcactgtacttatcgcttttatgattcatatcctgtcgacacacccacacaccctaggttaaagtgggaggggctgtaatgtacaagtatgtaaagtgtcggcaaaattagctcactgaccattcttagagacagaattcatctgagaagtctacatcagtggtctacttaccatcagaagcctgctatcaacccaataatga

SEQ ID NO: 3 - DNA, 24 bp, synthetic construct
  source 1..24: mol_type = other DNA
gttcattagttcccctgtgctgac

SEQ ID NO: 4 - DNA, 24 bp, synthetic construct
  source 1..24: mol_type = other DNA
tcattattgggttgatagcaggct

SEQ ID NO: 5 - DNA, 36 bp, synthetic construct
  source 1..36: mol_type = other DNA
acactctttccctacacgacgctgttccgatctnnn

SEQ ID NO: 6 - DNA, 13 bp, synthetic construct
  source 1..13: mol_type = other DNA
agatcggaacagc

SEQ ID NO: 7 - DNA, 35 bp, synthetic construct
  source 1..35: mol_type = other DNA
gtgactggagttcagacgtgtgctcttcacgannn

SEQ ID NO: 8 - DNA, 13 bp, synthetic construct
  source 1..13: mol_type = other DNA
tcgtgaagagcac

SEQ ID NO: 9 - DNA, 34 bp, synthetic construct
  source 1..34: mol_type = other DNA
acactctttccctacacgacgctcttcatcgnnn

SEQ ID NO: 10 - DNA, 13 bp, synthetic construct
  source 1..13: mol_type = other DNA
cgatgaagagcgt

SEQ ID NO: 11 - DNA, 35 bp, synthetic construct
  source 1..35: mol_type = other DNA
gtgactggagttcagacgtgtgctcttcagcannn

SEQ ID NO: 12 - DNA, 13 bp, synthetic construct
  source 1..13: mol_type = other DNA
tgctgaagagcac

SEQ ID NO: 13 - DNA, 34 bp, synthetic construct
  source 1..34: mol_type = other DNA
acactctttccctacacgacgctcttcatgcnnn

SEQ ID NO: 14 - DNA, 13 bp, synthetic construct
  source 1..13: mol_type = other DNA
gcatgaagagcgt

SEQ ID NO: 15 - DNA, 35 bp, synthetic construct
  source 1..35: mol_type = other DNA
gtgactggagttcagacgtgtgctcttcagacnnn

SEQ ID NO: 16 - DNA, 13 bp, synthetic construct
  source 1..13: mol_type = other DNA
gtctgaagagcac

SEQ ID NO: 17 - DNA, 34 bp, synthetic construct
  source 1..34: mol_type = other DNA
acactctttccctacacgacgctcttcagtcnnn

SEQ ID NO: 18 - DNA, 13 bp, synthetic construct
  source 1..13: mol_type = other DNA
gactgaagagcgt

SEQ ID NO: 19 - DNA, 35 bp, synthetic construct
  source 1..35: mol_type = other DNA
gtgactggagttcagacgtgtgctcttcacagnnn

SEQ ID NO: 20 - DNA, 13 bp, synthetic construct
  source 1..13: mol_type = other DNA
ctgtgaagagcac

SEQ ID NO: 21 - DNA, 34 bp, synthetic construct
  source 1..34: mol_type = other DNA
acactctttccctacacgacgctcttcactgnnn

SEQ ID NO: 22 - DNA, 13 bp, synthetic construct
  source 1..13: mol_type = other DNA
cagtgaagagcgt

SEQ ID NO: 23 - DNA, 37 bp, synthetic construct
  source 1..37: mol_type = other DNA
gtgactggagttcagacgtgtgctgttccgatctnnn

SEQ ID NO: 24 - DNA, 13 bp, synthetic construct
  source 1..13: mol_type = other DNA
agatcggaacagc

SEQ ID NO: 25 - DNA, 23 bp, synthetic construct
  source 1..23: mol_type = other DNA
acactctttccctacacgacgct

SEQ ID NO: 26 - DNA, 24 bp, synthetic construct
  source 1..24: mol_type = other DNA
gtgactggagttcagacgtgtgct

SEQ ID NO: 27 - DNA, 23 bp, synthetic construct
  source 1..23: mol_type = other DNA
acactctttccctacacgacgct

SEQ ID NO: 28 - DNA, 24 bp, synthetic construct
  source 1..24: mol_type = other DNA
gtgactggagttcagacgtgtgct

SEQ ID NO: 29 - DNA, 68 bp, synthetic construct
  source 1..68: mol_type = other DNA
aatgatacggcgaccaccgagatctacacnnnnnnacactctttccctacacgacgctcttccgatct

SEQ ID NO: 30 - DNA, 64 bp, synthetic construct
  source 1..64: mol_type = other DNA
caagcagaagacggcatacgagatnnnnnngtgactggagttcagacgtgtgctcttccgatct

SEQ ID NO: 31 - DNA, 24 bp, synthetic construct
  source 1..24: mol_type = other DNA
gttcattagttcccctgtgctgac

SEQ ID NO: 32 - DNA, 24 bp, synthetic construct
  source 1..24: mol_type = other DNA
tcattattgggttgatagcaggct